(19) United States
(12) Patent Application Publication
(10) Pub. No.: US 2024/0254440 A1
(43) Pub. Date: Aug. 1, 2024

(54) METHODS TO GENERATE GLIAL RESTRICTED NEURAL PROGENITOR CELLS AND USES THEREOF

(71) Applicant: The Regents of the University of California, Oakland, CA (US)

(72) Inventor: Alysson Renato Muotri, San Diego, CA (US)

(21) Appl. No.: 18/561,242

(22) PCT Filed: Jun. 30, 2022
(86) PCT No.: PCT/US2022/035841

§ 371 Date: Nov. 15, 2023

Related U.S. Application Data
(60) Provisional application No. 63/217,273, filed on Jun. 30, 2021.

(51) Int. Cl. C12N 5/0797; A61K 35/30

(52) U.S. Cl. CPC C12N 5/0623; A61K 35/30; C12N 2501/115; C12N 2501/727; C12N 2506/45; C12N 2513/00; C12N 2533/52

(57) ABSTRACT

The disclosure is directed to a method to generate glial restricted neural progenitor cells (GRNPCs), and uses thereof, including for the treatment of neurological or neurodegenerative disorders.

### CROSS REFERENCE TO RELATED APPLICATIONS

This application claims priority under 35 U.S.C. § 119 from Provisional Application Ser. No. 63/217,273, filed Jun. 30, 2021, the disclosure of which is incorporated herein by reference.

### TECHNICAL FIELD

The disclosure is directed to a method for generating glial restricted neural progenitor cells (GRNPCs), and uses thereof, including for the treatment of neurological or neurodegenerative disorders.

### BACKGROUND

Rett Syndrome or (RTT) is a devastating disease that effects every 1 in 10,000 children born in the United States, the majority of cases affecting mostly females. RTT is caused by mutations in the X-linked gene Methyl-CpG-binding Protein 2 (MECP2). This gene encodes the transcriptional regulator protein MeCP2. Currently, there are no cures or disease-specific treatments that are effective in treating those with RTT.

### SUMMARY

Both astrocytes and microglia dysfunction are known to be associated with many models of neurological diseases. In the studies presented herein, it was shown that astrocytes played an important role in RTT. The data presented herein clearly demonstrated that MECP2-mutant neurons can be rescued, both at morphological and functional levels, when surrounded by healthy human astrocytes. The data presented herein therefore indicate that mutations in the MECP2 gene are not deterministic, and that mutant neurons can be rescued in the environment provided by healthy astrocytes. Transplantation of fully differentiated human astrocytes is challenging, however, due to the sensitivity of the differentiated astrocytes to cultivation and injection stressors. As such, the use of differentiated human astrocytes for treatment options is negatively impacted by the difficulty in producing large numbers of such astrocytes. The methods presented herein overcome the foregoing production challenges by allowing for the expansion of GRNPCs in large quantities. Further, based upon the studies presented herein, the GRNPCs have utility in the treatment of neurological or neurodegenerative disorders.

In a particular embodiment, the disclosure provides a method to make glial restricted progenitor cells (GRNPCs) comprising: generating embryonic bodies (EBs) from ESCs or iPSCs by culturing the ESCs or iPSCs in a feeder-free maintenance medium and then in a SM1 neural induction medium; generating neural progenitor cells (NPCs) from EBs by plating the EBs onto laminin/polyornithine coated plates and culturing in SM1 neural induction media supplemented with bFGF; differentiating the NPCs to GRNPCs by: culturing the EBs in microwell culture plates in SM1 neural induction media supplemented with bFGF to form cell aggerates; collecting and culturing the cell aggregates for at least 24 h in suspension and under agitation to form free-floating spheres; culturing the spheres in SM1 neural induction media supplemented with a ROCK inhibitor for at least 24 h, and then in SM1 neural induction media for at least 7 days; culturing the spheres in astrocyte growth media for at least 10 days to form astrospheres; plating the astrospheres on laminin/polyornithine coated plates and culturing the plates for at least 5 days to form GRNPCs. In a further embodiment, the ESCs or iPSCs are human ESCs or human iPSCs. In yet a further embodiment, the human ESCs or human iPSCs are from a subject who does not have a neurological or neurodegenerative disorder. In another embodiment, the neurological or neurodegenerative disorder is Rett Syndrome. In a further embodiment, the feeder-free maintenance medium comprises DMEM/F12, FGF, TGFβ1, pipecolic acid, GABA, LiCl, lipid concentrate, L-glutamine-BME, MEM NEAA, and NaHCO₃. In yet a further embodiment, the SM1 neural induction media comprises DMEM/F12, insulin, transferring, progesterone, putrescine, selenium, vitamins, fatty acids, glutathione, pyruvate, catalase and superoxide dismutase. In a further embodiment, the ROCK inhibitor is selected from Y-30141, Y-33075, Y-39983, TCS-7001, verosudil, thiazovivin, RKI-1447, LX-7101, H-1152, GSK-576371, AT-13148, BA-210, and Y-27632. In a certain embodiment, the ROCK inhibitor is Y-27632. In another embodiment, the Astrocyte Growth Media comprises basal media supplemented with growth factors, cytokines, and supplements for Astrocyte growth.

The disclosure also provides a pure or substantially pure population of GRNPC obtained by the method of the disclosure.

The disclosure also provides a composition comprising a pharmaceutically acceptable carrier and the substantially pure population of cells of the disclosure.

In a certain embodiment, the disclosure further provides a method of treating a subject having a neurodegenerative or neurological disorder comprising: transplanting GRNPCs made by a method of the disclosure into neural tissue of a subject having a neurodegenerative or neurological disorder that is associated with astrocyte dysfunction. In a further embodiment, the neurodegenerative or neurological disorder that is associated with astrocyte dysfunction is selected from the group consisting of Rett Syndrome, Alexander's disease, amyotrophic lateral sclerosis (ALS), epilepsy, Parkinson's disease, Alzheimer's disease, and autism. In yet a further embodiment, the neurodegenerative or neurological disorder that is associated with astrocyte dysfunction is Rett Syndrome.

The disclosure also provides a method of treating a subject having a neurodegenerative or neurological disorder comprising: transplanting the substantially pure population of cells of the disclosure or the pharmaceutical composition of the disclosure into neuronal tissue of a subject having a neurodegenerative or neurological disorder that is associated with astrocyte dysfunction. In one embodiment, the neurodegenerative or neurological disorder that is associated with astrocyte dysfunction is selected from the group consisting of Rett Syndrome, Alexander's disease, amyotrophic lateral sclerosis (ALS), epilepsy, Parkinson's disease, Alzheimer's disease, and autism. In a further embodiment, the neurodegenerative or neurological disorder that is associated with astrocyte dysfunction is Rett Syndrome.

The disclosure also provides a method for treating a subject with a disease associated with malfunctioning astrocytes or glial cells, wherein the method comprises: transplanting GRNPC cells produced by the method of the disclosure, the cell population of the disclosure or the pharmaceutical composition of the disclosure to an affected neuronal location of a subject, wherein the transplanted cells rescue or restore glial cell and/or neuronal function, thereby treating the subject with the disease associated with malfunctioning glial cells. In one embodiment, the method includes generating embryonic bodies (EBs) from ESCs or iPSCs by culturing the ESCs or iPSCs in a feeder-free maintenance medium and then in a SM1 neural induction medium; generating neural progenitor cells (NPCs) from EBs by plating the EBs onto laminin/polyornithine coated plates and culturing in SM1 neural induction media supplemented with bFGF; differentiating the NPCs to GRNPCs by: culturing the EBs in microwell culture plates in SM1 neural induction media supplemented with bFGF to form cell aggerates; collecting and culturing the cell aggregates for at least 24 h in suspension and under agitation to form free-floating spheres; culturing the spheres in SM1 neural induction media supplemented with a ROCK inhibitor for at least 24 h, and then in SM1 neural induction media for at least 7 days; culturing the spheres in astrocyte growth media for at least 10 days to form astrospheres; plating the astrospheres on laminin/polyornithine coated plates and culturing the plates for at least 5 days to form GRNPCs and then implanting the GRNPCs. In one embodiment, the subject is a mammal. In a further embodiment, the mammal is human, rat, dog, cat, pig, horse, rabbit, cow, monkey or mouse.

### DESCRIPTION OF DRAWINGS

FIG. 1 diagrams a proposed roadmap for RTT. Use of cell transplantation for a neurodevelopmental disorder is proposed herein. In this schematic roadmap, stem cells are a resource tool (modified from RSRT).

FIG. 2A-E presents innovative tools to study iPSC-derived neurons. (A) Visualizing newly formed synapses from human neurons. The western blot is a validation of the Psd95-TS vector. Inhibition of the cleavage upon addition of BIL-2061. (B) Trafficking and accumulation of newly formed Psd95 molecules with time in synapses from iPSC-derived human cortical neurons. (C) Co-localization of pre- and post-synaptic terminals in live human neurons. (D) Lack of neuronal synchronization in RTT-derived cortical neurons using the MED64 MEA (5 minutes recordings, raster plots from multiple channels were removed for simplicity). The magenta boxes show examples of neuronal synchronization burst activity in at least four independent channels. (E) Significant reduction of synchronized neuronal burst events in RTT networks can be rescued by fixing the MECP2 mutation with CRISPR/Cas9 genome editing. Data shown as mean±SEM (n=3).

FIG. 3A-F presents the phenotypes in RTT-astrocytes. (A) Misregulation of astrocyte markers, secreted factors and glutamate pathway-related gene expression in RTT astrocytes by quantitative PCR. (B) Representative example of reduced calcium wave propagation in RTT-astrocytes compared to controls (WT). (C) Significant slow calcium wave spreading in RTT astrocytes upon stimuli. (D) Defects in glutamate clearance from RTT astrocytes over time. Red line shows the initial glutamate in the culture media in 0 h. (E) Differential release of cytokines in RTT astrocytes compared to controls by qPCR. (F) Validation of unbalanced cytokine release by RTT astrocytes in culture media by ELISA. Data shown as mean±SD (n=3; *p<0.01, **p<0.001 and ***p<0.0001).

FIG. 4A-D shows the co-culture of iPSC-derived neurons and astrocytes. (A) Schematic illustration of the protocol to generate neural progenitor cells (NPCs) and differentiate into purified neurons and astrocytes for co-culture assays. (B) Representative image of human neurons (MAP2) co-cultured on top of astrocytes (GFAP). (C) Representative images of control (WT) or RTT neurons (green) sorted in co-culture experiments. (D) RTT astrocytes are detrimental to neuronal maturation and the presence of WT astrocytes was enough to rescue several neuronal phenotypes observed in RTT, such as number of dendrites and neuronal length. Data shown as mean±SEM (n=25; *p<0.01).

FIG. 5 shows the functional rescue of RTT neurons. Co-culture assays revealed that healthy control (Ctrl) astrocytes can rescue synaptogenesis measured by co-localization of excitatory pre- and post-synaptic markers (top panel), and spike number (bottom panel) of RTT-derived neurons. Wells with only astrocytes did not exhibit any activity. Data shown as mean±SEM (n=20 neurons for puncta co-localization/50 um), and n=4 independent MEA wells).

FIG. 6A-E shows the Impact of MECP2 on human cortical organoids. (A) MECP2-knockout isogenic neurons (MECP2-KO) show reduced spine-like density and soma size compared to controls. (B) Organoid diameter quantification (CTR, n=210 organoids; KO, n=333 organoids). (C) Spine-like density and (D) synaptic puncta are reduced in MECP2-KO neurons. Scale bar, 50 μm. (E) Lack of oscillatory network events in 5-month-old MECP2-KO organoids. Data are shown as mean±s.e.m.; *p<0.05, **p<0.01, ***p<0.001, unpaired Student's t-test.

FIG. 7A-D shows human GFAP-positive astrocytes transplanted into mouse brains. (A) Isolated adult mouse brain with intact anatomy 3 months after cell transplantation. Arrow point to the original site of injection. (B) Distribution of the transplanted cells visualized with Trypan blue dye, 15 minutes after surgery showing correct injection site localization. (C) Widespread human astrocytes after 1-month post-transplantation of GRNPCs. (D) Detailed human astrocyte morphology in the mouse brain.

FIG. 8A-B shows healthy GRNPC transplantation in control brain organoids. (A) Live image of healthy dyed GRNPCs (green) in unaffected (WT) and MECP2 KO brain organoids 15 days post transplantation. (B) Transplanted GRNPCs integrate and differentiate into mature astrocytes.

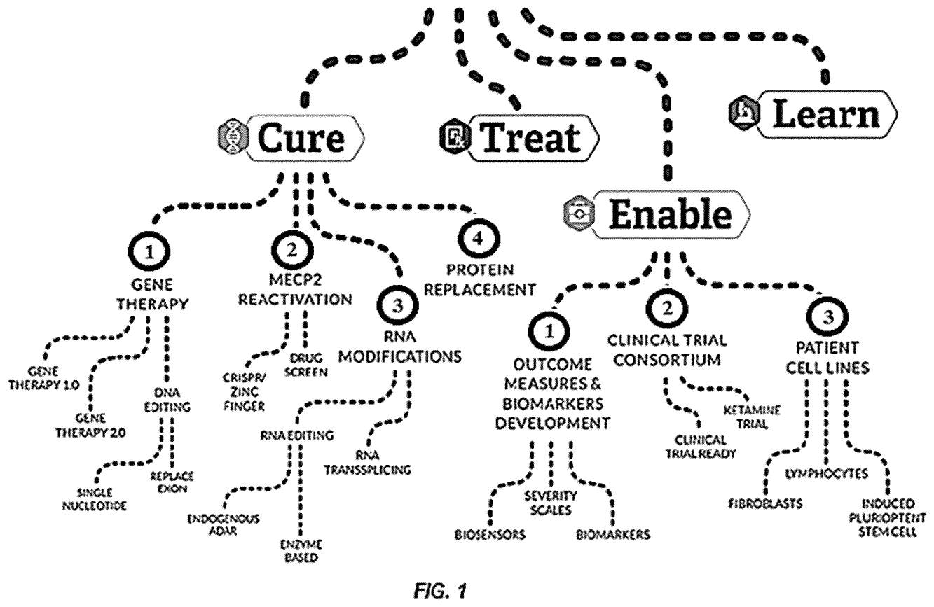

### DETAILED DESCRIPTION

As used herein and in the appended claims, the singular forms “a,” “an,” and “the” include plural referents unless the context clearly dictates otherwise. Thus, for example, reference to “a cell” includes a plurality of such cells and reference to “the mutation” includes reference to one or more mutations known to those skilled in the art, and so forth.

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

Also, the use of “or” means “and/or” unless indicated otherwise, such as by the use of the term “either.” Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

All publications mentioned herein are incorporated by reference in full for the purpose of describing and disclosing methodologies that might be used in connection with the description herein. Moreover, with respect to any term that is presented in the publications that is similar to, or identical with, a term that has been expressly defined in this disclosure, the definition of the term as expressly provided in this disclosure will control in all respects.

“Astrocyte” can refer to characteristic star-shaped glial cells in the brain and spinal cord. As would be clear to one skilled in the art, astrocytes can be characterized in their star shape, expression of markers like glial fibrillary acidic protein (GFAP) and aldehyde dehydrogenase 1 family member L1 (ALDH1L1), excitatory amino acid transporter 1/glutamate aspartate transporter (EAAT1/GLAST), glutamine synthetase, S100 beta, or excitatory amino acid transporter 1/glutamate transporter 1 (EAAT2/GLT-1), participation of blood-brain barrier together with endothelial cells, transmitter uptake and release, regulation of ionic concentration in extracellular space, reaction to neuronal injury and participation in nervous system repair, and metabolic support of surrounding neurons. In certain embodiments of the disclosure, an astrocyte can refer to a non-neuronal cell in a nervous system that expresses glial fibrillary acidic protein (GFAP), Aldehyde Dehydrogenase 1 Family Member L1 (ALDH1L1), or both. In certain embodiments, an astrocyte can refer to a non-neuronal cell in a nervous system that expresses a glial fibrillary acidic protein (GFAP) promoter-driven transgene (e.g., red fluorescent protein (RFP), Cre recombinase).

A “chemically defined” medium or “chemically defined” culture medium is a culture medium whose components are chemically defined. As such, it lacks serum, a chemically undefined component. For culturing of cells or tissues requiring serum, a “chemically defined” medium typically contains serum substitute or serum replacement in place of serum. A “chemically defined” medium is “xeno-free” if it contains no animal-derived product or no foreign animal derived product. It may also be free of human-derived product. It is generally desirable to replace animal- or human-derived products with recombinantly produced materials, chemically synthesized materials, or enzymatically synthesized materials, which have no prior animal contact or exposure

As used herein, the term “contacting” cells with a composition of the disclosure refers to placing the composition (e.g., compound, nucleic acid, viral vector etc.) in a location that will allow it to touch the cell in order to produce “contacted” cells. The contacting may be accomplished using any suitable method. For example, in one embodiment, contacting is by adding the compound to a culture of cells. Contacting may also be accomplished by injecting it or delivering the composition to a location within a body such that the composition “contacts” the cell type targeted.

The term “de-differentiation” is familiar to the person skilled in the relevant art. In general, de-differentiation signifies the regression of lineage committed cell to the status of a stem cell, for example, by “inducing” a de-differentiated phenotype. For example, as described further herein KLF4, OCT4, SOX2, c-MYC or n-MYC, and Nanog can induce de-differentiation and induction of mitosis in lineage committed mitotically inhibited cells.

As used herein, the term “differentiation”, of “differentiate” or “converting” of “inducing differentiation” are used interchangeably to refer to changing the default cell type (genotype and/or phenotype) to a non-default cell type (genotype and/or phenotype). Thus “inducing differentiation in an astrocyte cell” refers to inducing the cell to change its morphology from an astrocyte to a neuronal cell type (i.e., change in gene expression as determined by genetic analysis such as a microarray) and/or phenotype (i.e. change in expression of a protein.

A “feeder-free culture medium” refers to the fact that the culture medium used to culture the cells of interest do not need feeder cells in order to permit the cells to proliferate stably.

As used herein, the term “glial cell” can generally refer to a type of supportive cell in the central nervous system (e.g., brain and spinal cord) and the peripheral nervous system. In some embodiments, unlike neurons, glial cells do not conduct electrical impulses or exhibit action potential. In some embodiments, glial cells do not transmit information with each other, or with neurons via synaptic connection or electrical signals. In a nervous system or in an in vitro culture system, glial cells can surround neurons and provide support for and insulation between neurons. Non-limiting examples of glial cells include oligodendrocytes, astrocytes, ependymal cells, Schwann cells, microglia, and satellite cells.

As used herein, the term “neuron” or “neuronal cell” as used herein can have the ordinary meaning one skilled in the art would appreciate. In some embodiments, neuron can refer to an electrically excitable cell that can receive, process, and transmit information through electrical signals (e.g., membrane potential discharges) and chemical signals (e.g., synaptic transmission of neurotransmitters). As one skilled in the art would appreciate, the chemical signals (e.g., based on release and recognition of neurotransmitters) transduced between neurons can occur via specialized connections called synapses. As used herein, the term “mature neuron” can refer to a differentiated neuron. In some embodiments, a neuron is the to be a mature neuron if it expresses one or more markers of mature neurons, e.g., microtubule-associated protein 2 (MAP2) and Neuronal Nuclei (NeuN), neuron specific enolase (NSE), 160 kDa neurofilament medium, 200 kDa neurofilament heavy, postsynaptic density protein 95 (PDS-95), Synapsin I, Synaptophysin, glutamate decarboxylase 67 (GAD67), glutamate decarboxylase 67 (GAD65), parvalbumin, dopamine- and CAMP-regulated neuronal phosphoprotein 32 (DARPP32), vesicular glutamate transporter 1 (vGLUT1), vesicular glutamate transporter 2 (vGLUT2), acetylcholine, and tyrosine hydroxylase (TH). As used herein, the term “functional neuron” can refer to a neuron that is able to send or receive information through chemical or electrical signals. In some embodiments, a functional neuron exhibits one or more functional properties of a mature neuron that exists in a normal nervous system, including, but not limited to: excitability (e.g., ability to exhibit action potential, e.g., a rapid rise and subsequent fall in voltage or membrane potential across a cellular membrane), forming synaptic connections with other neurons, pre-synaptic neurotransmitter release, and post-synaptic response (e.g., excitatory postsynaptic current or inhibitory postsynaptic current). In some embodiments, a functional neuron is characterized in its expression of one or more markers of functional neurons, including, but not limited to, synapsin, synaptophysin, glutamate decarboxylase 67 (GAD67), glutamate decarboxylase 67 (GAD65), parvalbumin, dopamine- and CAMP-regulated neuronal phosphoprotein 32 (DARPP32), vesicular glutamate transporter 1 (vGLUT1), vesicular glutamate transporter 2 (vGLUT2), acetylcholine, tyrosine hydroxylase (TH), dopamine, vesicular GABA transporter (VGAT), and gamma-aminobutyric acid (GABA).

As used herein, the term “non-neuronal cell” can refer to any type of cell that is not a neuron. An exemplary non-neuronal cell is a cell that is of a cellular lineage other than a neuronal lineage (e.g., a hematopoietic lineage). In some embodiments, a non-neuronal cell is a cell of neuronal lineage but not a neuron, for example, a glial cell. In some embodiments, a non-neuronal cell is somatic cell that is not neuron, such as, but not limited to, glial cell, adult primary fibroblast, embryonic fibroblast, epithelial cell, melanocyte, keratinocyte, adipocyte, blood cell, bone marrow stromal cell, Langerhans cell, muscle cell, rectal cell, or chondrocyte. In some embodiments, a non-neuronal cell is from a non-neuronal cell line, such as, but not limited to, glioblastoma cell line, Hela cell line, NT2 cell line, ARPE19 cell line, or N2A cell line. “Cell lineage” or “lineage” can denote the developmental history of a tissue or organ from the fertilized embryo. “Neuronal lineage” can refer to the developmental history from a neural stem cell to a mature neuron, including the various stages along this process (as known as neurogenesis), such as, but not limited to, neural stem cells (neuroepithelial cells, radial glial cells), neural progenitors (e.g., intermediate neuronal precursors), neurons, astrocytes, oligodendrocytes, and microglia.

The terms “nucleic acid” and “polynucleotide” as used interchangeably herein can refer to deoxyribonucleotides or ribonucleotides and polymers thereof in either single- or double-stranded form. The term can encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, locked nucleic acids (LNAs), and peptide-nucleic acids (PNAs).

“Oligodendrocyte” can refer to a type of glial call that can create myelin sheath that surrounds a neuronal axon to provide support and insulation to axons in the central nervous system. Oligodendrocyte can also be characterized in their expression of PDGF receptor alpha (PDGFR-α), SOX10, neural/glial antigen 2 (NG2), Olig 1, 2, and 3, oligodendrocyte specific protein (OSP), Myelin basic protein (MBP), or myelin oligodendrocyte glycoprotein (MOG).

The term “precursor cell,” “progenitor cell,” and “stem cell” are used interchangeably in the art and herein and refer either to a pluripotent, or lineage-uncommitted, progenitor cell, which is potentially capable of an unlimited number of mitotic divisions to either renew its line or to produce progeny cells, which will differentiate into fibroblasts or a lineage-committed progenitor cell and its progeny, which is capable of self-renewal and is capable of differentiating into a parenchymal cell type. Unlike pluripotent stem cells, lineage-committed progenitor cells are generally considered to be incapable of giving rise to numerous cell types that phenotypically differ from each other. Instead, they give rise to one or possibly two lineage-committed cell types.

The terms “protein,” “peptide,” and “polypeptide” as used interchangeably can refer to an amino acid polymer or a set of two or more interacting or bound amino acid polymers.

The term “promoter,” as used herein, can refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. Promoters include constitutive and inducible promoters. A “constitutive” promoter is a promoter that can be active under most environmental and developmental conditions. An “inducible” promoter is a promoter that can be active under environmental or developmental regulation. The term “operably linked” can refer to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

As used herein, the term “reprogramming” or “trans-differentiation” can refer to the generation of a cell of a certain lineage (e.g., a neuronal cell) from a different type of cell (e.g., a fibroblast cell) without an intermediate process of de-differentiating the cell into a cell exhibiting pluripotent stem cell characteristics. “Pluripotent” can refer to the ability of a cell to form all lineages of the body or soma (i.e., the embryo proper). Exemplary “pluripotent stem cells” can include embryonic stem cells and induced pluripotent stem cells.

“Serum-free” refers to a lack of serum, which is a clarified blood product obtained from an animal or human.

A “serum-free” culture medium is a culture medium lacking serum. It may contain, for example, serum substitute or serum replacement.

The terms “subject” and “patient” as used interchangeably can refer to, except where indicated, mammals such as humans and non-human primates, as well as rabbits, rats, mice, goats, pigs, and other mammalian species. The term does not necessarily indicate that the subject has been diagnosed with a particular disease, but instead can refer to an individual under medical supervision. For example, mammalian species that benefit from the disclosed methods and composition include, but are not limited to, primates, such as apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo bison, horses, donkey, swine, sheep, and goats; exotic animals typically found in zoos such as bear, lions, tigers, panthers, elephants, hippopotamus, rhinoceros, giraffes antelopes, sloth, gazelles, zebras, wildebeests, prairie dogs, koala bears, kangaroo opossums, raccoons, pandas, hyena, seals, sea lions, elephant seals, otters, porpoises dolphins, and whales.

The term “transplantation” as used herein refers to the administration of a composition comprising cells, including a cell suspension or cells incorporated into a matrix or tissue, that are either in an undifferentiated, partially differentiated, or fully differentiated form, into a human or other animal.

A “vector” is a nucleic acid that can be capable of transporting another nucleic acid into a cell. A vector can be capable of directing expression of a protein or proteins encoded by one or more genes, or a microRNA encoded by a polynucleotide, carried by the vector when it is present in the appropriate environment.

A “viral vector” is a viral-derived nucleic acid that can be capable of transporting another nucleic acid into a cell. A viral vector can be capable of directing expression of a protein or proteins encoded by one or more genes, or a microRNA encoded by a polynucleotide, carried by the vector when it is present in the appropriate environment. Examples of viral vectors include, but are not limited to, retroviral, adenoviral, lentiviral and adeno-associated viral vectors.

Rett Syndrome (RTT) is a severe progressive neurological disorder caused by mutations in the X-linked gene Methyl-CpG-binding Protein 2 (MECP2), which encodes the transcriptional regulator protein MeCP2. There is currently no cure or disease-specific treatments for RTT. People with RTT undergo a distinct regression during the first years of life, losing the ability to speak and use their hands and then display repetitive hand movements. During regression, autistic features, such as social withdrawal and loss of eye contact, may be severe. As the behavioral and motor features of RTT appear around the same time as features for autism spectrum disorder (ASD) manifest, suggests a potential common underlying biological process.

Mouse models of RTT reproduce many of the clinical features observed in people with RTT. These mouse models have contributed to many insights about the condition. However, there are numerous intrinsic interspecies differences that result in large gaps of knowledge about unique aspects of the condition in humans. A non-cell autonomous effect of astrocytes carrying MECP2 mutations was first described in vitro, then in vivo and recently from human cells. However, how MeCP2 regulates the molecular mechanism by which astrocytes affect neuronal homeostasis is still unknown. Astrocytes play a major role within the central nervous system (CNS) by having numerous functions, including glutamate, ion, and water homeostasis; defense against oxidative/nitrosative stress; energy storage; mitochondria biogenesis; scar formation; and tissue repair. In order to systematically evaluate the contribution of astrocytes to neuronal function, RTT-iPSCs were generated to re-create controlled cultures of human neurons and astrocytes in vitro. Using a specially designed protocol, human glial-restricted neural progenitor cells (GRNPCs) were generated from iPSCs. The interplay between neurons and astrocytes were then studied in vitro. Healthy neurons co-cultured atop healthy astrocytes show vibrant dendritic arborization, a feature lost when the neurons were co-cultured with RTT-derived astrocytes. This suggests a dominant-negative effect of RTT astrocytes on previously healthy neurons. Surprisingly, RTT neurons co-cultured in the presence of healthy astrocytes reverted all neuronal alterations, indicating the possibility that transplantation of healthy glial progenitor cells could be beneficial to RTT. Accordingly, the disclosure provides a method of treating a subject having a neurodegenerative or neurological disorder comprising: transplanting GRNPCs made by a method of the disclosure into neural tissue of a subject having a neurodegenerative or neurological disorder that is associated with astrocyte dysfunction. Examples of neurodegenerative or neurological disorders that are associated with astrocyte dysfunction include, but are not limited to, Rett Syndrome, Alexander's disease, amyotrophic lateral sclerosis (ALS), epilepsy, Parkinson's disease, Alzheimer's disease, autism spectrum disorders and autism. In a particular embodiment, the neurodegenerative or neurological disorder that is associated with astrocyte dysfunction is Rett Syndrome.

Autism Spectrum Disorder (ASD) is a serious developmental disorder that impairs the ability to communicate or interact with others. Autism Spectrum Disorder (ASD) impacts the nervous system. Autism Spectrum Disorder (ASD) has a wide range of severity and of symptoms. Common symptom of autism spectrum disorder (ASD) include: Difficulty communicating, difficulty with social behavior/interactions, obsessive interests, and repetitive behaviors.

Alexander Disease is a disorder that effects the nervous system. It is one of a group of disorder called leukodystrophies that involve the destruction of myelin. Myelin being the fatty covering that insulates nerve fibers and which also promotes the rapid transmission of nerve impulses. If the myelin is not properly maintained, the transmission of nerve impulses could be disrupted. As myelin deteriorates in leukodystrophies, nervous system function is impaired.

Amyotrophic Lateral Sclerosis (ALS) is a neurological disease that primarily affects the nerve cells (neurons) responsible for controlling voluntary muscle movement. Voluntary muscles produce movements like chewing, walking, and talking. The disease is a progressive, meaning the symptoms get worse over time. Amyotrophic Lateral Sclerosis (ALS) belongs to a wider group of disorders known as motor neuron diseases. The cause of these diseases are from gradual deterioration (degeneration) and death of motor neurons.

Alzheimer's disease is a progressive mental deterioration that can occur in middle or old age, due to generalized degeneration of the brain. Alzheimer's disease is a type of dementia that affects memory, thinking and behavior. Alzheimer's slowly progresses where dementia symptoms gradually worsen over several years. In the early stages of Alzheimer's disease may be seen as slight or mild memory loss with the symptoms gradually becoming worse overtime. Those diagnosed with Alzheimer's have an average lifespan of 4-8 years after diagnoses.

Epilepsy is a chronic seizure disorder of the nervous system. In individuals with Epilepsy, the nerve cells in the brain fire electrical impulses at a rate of up to four times higher than normal, this causes a sort of electrical storm in the brain, known as a seizure. Known causes of Epilepsy include: brain injuries, brain tumors, lead poisoning, maldevelopment of the brain, genetic and infectious illnesses. Epilepsy can be characterized as mild (Petit mal) or severe (Grand mal).

Parkinson's disease is a brain disorder that leads to shaking, stiffness, and difficulty with walking, balance, and coordination. This disorder affects the central nervous system, caused by nerve cell damage in the brain which causes dopamine levels to drop, leading to symptoms.

Rett Syndrome (RTT) is a devastating disease that affects 1 in every 10,000 children, primarily females, in the United States. Rett Syndrome (RTT) is a severe neurological disorder caused by mutations in the X-linked methyl-CpG-binding protein 2 (MeCP2) gene. Although previous studies suggest that astrocytes play an important role in neuronal homeostasis, how the absence of MeCP2 in human astrocytes affect neuronal development is unknown. Currently, there are no known preventative approaches or therapeutic treatments available for RTT. RTT patients undergo seemingly normal development until 6-18 months of age, where the onset of motor impairments, stagnation, regression of developmental skills, hypotonia, seizures and a spectrum of autistic behaviors begin to occur. The development of autistic features, such as social withdrawal and loss of eye contact, characterize the behavioral and motor regression phase of RTT. Alterations in the MeCP2 gene clearly impair the physiology of human neurons, and experiments using mouse models, and iPSC-derived mouse astrocytes indicate that the loss of MeCP2 protein function in astrocytes negatively affects neurons in a non-cell-autonomous fashion.

Clinical similarities in behavior phenotypes between the regressive phase in RTT and other ASD patients suggest a common underlying biological process. One important link between ASD and RTT is an imbalance in cytokine signaling. IL-6 has been associated with ASD in mouse models; and a single injection of IL-6 in a pregnant mouse is sufficient to produce offspring with autistic features. Furthermore, IL-6 has been linked to alterations in ASD patients' brains, affecting neural cell adhesion, migration, and synapse maturation. Previous experiments investigating the cell types responsible for the detrimental effects of IL-6 in the brain, showed that selective IL-6 overexpression in mouse astrocytes revealed robust ASD behavioral features. Immunological impairments have been reported in RTT, specially focusing on interleukin abnormalities, which include elevated levels of IL-6. Importantly, cytokine signaling can differ between mice and humans, and the ability to rapidly profile different mutations in relevant cell types highlights the value of the human iPSC system to complement current disease modeling efforts. Further investigation of the mechanisms involved in the release of signaling cytokines and their regulation by MeCP2 in glial cells is necessary for a better understanding of RTT pathology. The efficient astrocyte-differentiation method described here, together with the insights on RTT astrocytes, can be applied to other neurological disorders with a non-cell autonomous neuro-inflammatory component.

The disclosure provides a method to make glial restricted progenitor cells (GRNPCs) from ESCs or iPSCs. Embryonic stem cells (ESC) are pluripotent cells obtained from the inner cell mass (ICM) of blastocysts derived from in vitro culture associated with reproductive endocrinology therapy. Human ESCs are regarded as highly significant since they retain the capacity to differentiate into any of approximately 200 unique cell types. Human ESCs are isolated from human blastocysts of human embryos and therefore are quite controversial. As such, a more preferred method for generating GRNPCs is from human iPSCs.

Human fibroblast (or other somatic cells) can be isolated from a subject and de-differentiated to induced pluripotent stem cells (iPSCs). The disclosure uses a plurality of de-differentiation factors for de-differentiating lineage committed cells to a more pluripotent or omnipotent cell type. As used herein a de-differentiation factor comprises a polynucleotide, polypeptide or small molecule. Exemplary de-differentiation factors comprising a polynucleotide are selected from the group consisting of a polynucleotide encoding a NANOG polypeptide, a c-MYC or n-MYC polypeptide, a KLF4 polypeptide, a SOX2 polypeptide or OCT4 polypeptide. Exemplary polypeptides comprise NANOG, c-MYC or n-MYC, KLF4, SOX2 or OCT4 polypeptides or polypeptides that increase the expression of any of the foregoing. Useful small molecule de-differentiation factors include molecules that stimulate the transcription or activity of an endogenous Nanog, c-Myc or n-Myc, Klf4, Sox4 or Oct4 polynucleotide or polypeptide, respectively.

A method to de-differentiate cells by expression of KLF4, OCT4, SOX2, c-MYC or n-MYC, NANOG or any combination thereof is presented. The nucleic acid and amino acid sequences of mouse and human KLF4, OCT4, SOX2, c-MYC or n-MYC, NANOG are known in the art (see, e.g., U.S. Pat. No. 9,005,966, which is incorporated by reference). The disclosure demonstrates that transfection with KLF4, OCT4, SOX2, c-MYC or n-MYC, and NANOG results in a de-differentiation of committed fibroblasts (e.g., dermal fibroblasts) to a pool of proliferating stem cells that are capable of re-differentiating into several cell types (including lineage committed neuronal cells).

In addition to the expression of a nucleic acid encoding an KLF4, OCT4, SOX2, c-MYC or n-MYC, and/or NANOG polypeptide, the disclosure contemplates that any agent which increase the expression and/or activity of an endogenous KLF4, OCT4, SOX2, c-MYC or n-MYC, NANOG or any combination thereof can be used in the methods of the disclosure to promote de-differentiation.

Nanog is a gene expressed in embryonic stem cells (ESCs) and plays a role in maintaining pluripotency. NANOG is thought to function with SOX2. Human NANOG protein (see, e.g., Accession number NP_079141, incorporated herein by reference) is a 305 amino acid protein with a homeodomain motif that is localized to the nuclear component of cells. Similar to murine NANOG, N-terminal region of human NANOG is rich in Ser, Thr and Pro residues and the C-terminus comprises Trp repeats. The homeodomain in human NANOG ranges from about residue 95 to about residue 155. Homologs of human NANOG are known.

Oct-4 (Octamer-4) is a homeodomain transcription factor of the POU family and regulates the expression of numerous genes (see, e.g., J. Biol. Chem., Vol. 282, Issue 29, 21551-21560, Jul. 20, 2007, incorporated herein by reference). Homologs of human Oct-4 are known as set forth in the following accession numbers NP_038661.1 and NM_013633.1 (Mus musculus), NP_001009178 and NM_001009178 (Rattus norvegicus), and NP_571187 and NM_131112 (Danio rerio), which are incorporated herein by reference.

SRY (sex determining region Y)-box 2, also known as SOX2, is a transcription factor that plays a role in self-renewal of undifferentiated embryonic stem cells and transactivation of Fgf4 as well as modulating DNA bending (see, e.g., Scaffidi et al. J. Biol. Chem., Vol. 276, Issue 50, 47296-47302, Dec. 14, 2001, incorporated herein by reference). Homologs of human SOX2 are known.

Kruppel-like factor 4, also known as KLF4 plays a role in stem cell maintenance and growth. Homologs of human KLF4 are known and include NP_034767, NM_010637 (Mus musculus), which are incorporated herein by reference.

The MYC family of cellular genes is comprised of c-myc, N-myc, and L-myc, three genes that function in regulation of cellular proliferation, differentiation, and apoptosis (Henriksson and Luscher 1996; Facchini and Penn 1998). Although myc family genes have common structural and biological activity. N-Myc is a member of the MYC family and encodes a protein with a basic helix-loop-helix (bHLH) domain. The genomic structures of C-myc and N-myc are similarly organized and are comprised of three exons. Most of the first exon and the 3′ portion of the third exon contain untranslated regions that carry transcriptional or post-transcriptional regulatory sequences. N-myc protein is found in the nucleus and dimerizes with another bHLH protein in order to bind DNA. Homologs and variants of the Myc family of proteins are known in the art.

CDNA coding for the human oct4 (pour5f1), sox2, klf4, c-myc (or n-myc) and nanog, variants and homologs thereof can be cloned and expressed using techniques known in the art. Using the sequences set forth in the accession numbers above and available to one of skill in the art, one or more de-differentiation factors can be cloned into a suitable vector for expression in a cell type of interest.

Cells can be engineered using any of a variety of vectors including, but not limited to, integrating viral vectors, e.g., retrovirus vector or adeno-associated viral vectors; or non-integrating replicating vectors, e.g., papilloma virus vectors, SV40 vectors, adenoviral vectors; or replication-defective viral vectors. Where transient expression is desired, non-integrating vectors and replication defective vectors may be used, since either inducible or constitutive promoters can be used in these systems to control expression of the gene of interest. Where the vector is a non-integrating vector, such vectors can be lost from cells by dilution after reprogramming, as desired. An example of a non-integrating vector includes Epstein-Barr virus (EBV) vector. Alternatively, integrating vectors can be used to obtain transient expression, provided the gene of interest is controlled by an inducible promoter. Other methods of introducing DNA into cells include the use of liposomes, lipofection, electroporation, a particle gun, or by direct DNA injection.

Conventional recombinant DNA techniques are used in the methods of the disclosure. For example, conventional recombinant DNA techniques are used to introduce the desired polynucleotide (e.g., KLF4, OCT4, SOX2, c-MYC or n-MYC, NANOG or any combination thereof) into differentiated cells to de-differentiate the cells into stem cells. The precise method used to introduce a polynucleotide is not critical to the disclosure. For example, physical methods for the introduction of polynucleotides into cells include microinjection and electroporation. Chemical methods such as co-precipitation with calcium phosphate and incorporation of polynucleotides into liposomes are also standard methods of introducing polynucleotides into mammalian cells. For example, DNA or RNA can be introduced using standard vectors, such as those derived from murine and avian retroviruses (see, e.g., Gluzman et al., 1988, Viral Vectors, Cold Spring Harbor Laboratory, Cold Spring Harbor, N.Y.). Standard recombinant molecular biology methods are well known in the art (see, e.g., Ausubel et al., 1989, Current Protocols in Molecular Biology, John Wiley & Sons, New York), and viral vectors for gene therapy have been developed and successfully used clinically (Rosenberg, et al., 1990, N. Engl. J. Med, 323: 370). Other methods, such as naked polynucleotide uptake from a matrix coated with DNA are also encompassed by the disclosure (see, for example, U.S. Pat. No. 5,962,427, which is incorporated herein by reference) or the use of alphaviral replicons (see, e.g., U.S. Pat. No. 9,862,930, which is incorporated herein by reference).

Somatic cells, such as fibroblasts, are transformed or transfected with a polynucleotide(s) encoding a de-differentiation factor(s) (or a combination of polynucleotide factors, proteins or small molecules), e.g., DNA, controlled by or in operative association with one or more appropriate expression control elements such as promoter or enhancer sequences, transcription terminators, polyadenylation sites, among others, and may further include a detectable marker. Following the introduction of the foreign DNA, engineered cells may be allowed to grow in enriched media and then switched to selective media.

Any promoter may be used to drive the expression of the inserted gene. For example, viral promoters include, but are not limited to, the CMV promoter/enhancer, SV40, papillomavirus, Epstein-Barr virus, elastin gene promoter and beta-globin. The control elements used to control expression of the polynucleotide encoding a de-differentiation factor should allow for the regulated expression of the polynucleotide. If transient expression is desired, constitutive promoters are used in a non-integrating and/or replication-defective vector. Alternatively, inducible promoters could be used to drive the expression of the inserted polynucleotide when necessary. Inducible promoters can be built into integrating and/or replicating vectors. For example, inducible promoters include, but are not limited to, metallothionein and promoters associated with heat shock proteins.

For example, the de-differentiation factors set forth herein can be cloned into an expression vector (e.g., a retroviral vector such as a pMX retroviral vector). The expression vector can be transformed into a cell of interest (e.g., a fibroblast). A de-differentiation factor can be introduced by transfection or transduction into a somatic cell or stromal cell using a vector, such as an integrating- or non-integrating vector. After introduction, the DNA segment(s) encoding the de-differentiation factor(s) can be extra-chromosomally (e.g., on an episomal plasmid) or stably integrated into cellular chromosome(s).

Where a retroviral vector is used a virus particle can be generated in a host cell to obtain infectious viral particles (e.g., in cell such as Phoenix-A cells). A cell-type of interest (e.g., a cell type to be de-differentiated) can then be infected with virus and cultured appropriately to select and grow the de-differentiated phenotype. For example, in one embodiment, human dermal fibroblasts are infected twice over 3 days at passage 6 and then re-plated four days later onto a feeder lay (e.g., an irradiated murine fibroblasts feeder layer). For studies involving regulation of genes associated with a disease, the fibroblast can be obtained from a subject having the particular disease. For example, in one embodiment, the human dermal fibroblasts are obtained from a subject having an RTT-associated mutation such that the resulting iPSCs carry the disease associated gene mutation.

The vector can include a single DNA segment encoding a single de-differentiation factor or a plurality of de-differentiation factors in any order so long as that they are operably expressed and function in a recombinant host cell. Where a vector includes one or some of the de-differentiation factors, but not all, a plurality of vectors (e.g., 2, 3, 4, or 5) can be introduced into a single somatic or stromal cell. A marker such as an expressed marker (e.g., a fluorescent protein such as GFP) can be used in combination with the de-differentiation factor to measure expression from the vector. For example, a GFP marker can be used to measure expression from a retroviral vector. The disclosure demonstrates that loss of expression from a retroviral vector comprising a de-differentiation factor can be used to select/enrich for stem cells. Alternatively, one or more markers of de-differentiation (e.g., pluripotent status) can be measured.

The vectors described herein can be constructed and engineered using art-recognized techniques to increase their safety for use in therapy and to include suitable expression elements and therapeutic genes. Standard techniques for the construction of expression vectors suitable for use as described herein are well-known to one of ordinary skill in the art and can be found in such publications such as Sambrook J, et al., “Molecular cloning: a laboratory manual,” (3rd ed. Cold Spring Harbor Press, Cold Spring Harbor, N.Y. 2001), incorporated herein by reference as if set forth in its entirety.

The somatic cells used to obtain induced pluripotent stem cells can be isolated from any number of various tissues of the body. For example, the sample of cells may be obtained from bone marrow, fetal tissue (e.g., fetal liver tissue), peripheral blood, umbilical cord blood, pancreas, skin and the like. In one embodiment, the cells are fibroblasts. In certain diagnostic and therapeutic discovery embodiment, the cells are obtained from a subject having, or suspected of having, a mutation that causes a neuronal disease or disorder (e.g., causing RTT syndrome or any other neurological disease or disorder). As is well known a somatic cell includes the genetic makeup of the individual and thus any induced pluripotent stem cell obtained from the somatic cell will include the same genetic makeup (e.g., same mutations found in the somatic cells obtained from the subject).

The methods of the disclosure may be applied to a procedure wherein differentiated (lineage committed) cells are removed from a subject, de-differentiated in culture, manipulated to re-differentiate along a specific differentiation pathway (e.g., neuronal cells) and then cultured and studied to (a) determine a mutations phenotypic result, (b) screen agents for their effect on the mutant dedifferentiated neuronal cell, or (c) as therapeutics.

For example, fibroblasts can be removed from a subject, de-differentiated using de-differentiation factors (e.g., with a KLF4, OCT4, SOX2, c-MYC or n-MYC, NANOG agonists or any combination thereof) and optionally mitotically expanded and then re-differentiated (e.g., with a KLF4, OCT4, SOX2, c-MYC or n-MYC, NANOG antagonists or any combination thereof) or factors (including physical stimuli) known to cause differentiation of hESCs down a lineage committed path. In one embodiment, the method comprises removing differentiated cells from an injured or diseased subject. Cells de-differentiated from cells harvested from a subject can then be studied to determine a suitable therapy and/or screened to identify drug/biological candidates of interest to treat the disease or disorder. In one embodiment, the “de-differentiated cells” are differentiated down a lineage committed path to study a particular disease. For example, the de-differentiated cells (e.g., iPSC) can be differentiated down a neuronal lineage to obtain, e.g., astrocytes.

The somatic cells used to obtain induced pluripotent stem cells can be isolated from any number of various tissues of the body. For example, the sample of cells may be obtained from bone marrow, fetal tissue (e.g., fetal liver tissue), peripheral blood, umbilical cord blood, pancreas, skin and the like. In one embodiment, the cells are fibroblasts and in a further embodiment, the cells are obtained from a subject having, or suspected of having, a mutation that causes a neuronal disease or disorder (e.g., causing RTT syndrome or any other neurological disease or disorder). As is well known a somatic cell includes the genetic makeup of the individual and thus any induced pluripotent stem cell obtained from the somatic cell will include the same genetic makeup (e.g., same mutations found in the somatic cells obtained from the subject).

The methods of the disclosure may be applied to a procedure wherein differentiated (lineage committed) cells are removed from a subject, de-differentiated in culture, manipulated to re-differentiate along a specific differentiation pathway (e.g., neuronal cells) and then cultured and studied to (a) determine a mutations phenotypic result and/or (b) screen agents for their effect on the mutant dedifferentiated neuronal cell.

For example, fibroblasts can be removed from a subject, de-differentiated using de-differentiation factors (e.g., with a KLF4, OCT4, SOX2, c-MYC or n-MYC, NANOG agonists or any combination thereof) and optionally mitotically expanded and then re-differentiated (e.g., with a KLF4, OCT4, SOX2, c-MYC or n-MYC, NANOG antagonists or any combination thereof) or factors (including physical stimuli) known to cause differentiation of hESCs down a lineage committed path. In one embodiment, the method comprises removing differentiated cells from an injured or diseased subject. Cells de-differentiated from cells harvested from a subject can then be studied to determine a suitable therapy and/or screened to identify drug/biological candidates of interest to treat the disease or disorder. In one embodiment, the “de-differentiated cells” are differentiated down a lineage committed path to study a particular disease. For example, the de-differentiated cells (e.g., iPSC) can be differentiated down a neuronal lineage to obtain, e.g., astrocytes.

The isolation of cells, such as fibroblasts, from a subject are known. For example, the isolation of fibroblasts may, for example, be carried out as follows: fresh tissue, e.g., a biopsy, samples are thoroughly washed and minced in Hanks balanced salt solution (HBSS) in order to remove serum. The minced tissue is incubated from 1-12 hours in a freshly prepared solution of a dissociating enzyme such as trypsin. After such incubation, the dissociated cells are suspended, pelleted by centrifugation and plated onto culture dishes. Fibroblasts cells will attach to the culture dish before other cells, therefore, appropriate stromal cells can be selectively isolated and grown.

Somatic cell (e.g., a population of somatic cells such as fibroblasts) are obtained from a subject are de-differentiated into induced pluripotent stem cells (iPSCs). For mutations having effects on neuronal processing and development, the iPSCs are then differentiated to neuronal cells. Because the genome of the iPSCs will carry the mutant gene, the differentiated neuronal cells will also carry the same mutation. In this way, the effect of the mutation on neuronal function can be studied. In addition, various factors or agents can be used to modulate the effect of the mutation on neuronal cell function, which may further be specific for the subject that was the source of the cells. In other words, the differentiated neuronal cells can be used to screen agents for effects on the biological function of the mutant neuronal cells. In this way, agents that show a beneficial effect on a particular mutation can then be advanced as potential therapeutics.

In one embodiment, a pluripotent stem cell (e.g., an iPSC) is differentiated into a neural progenitor cell (NPC) using dual SMAD inhibitor molecules. Exemplary embodiments include differentiating iPSC in the presence of Noggin (e.g., human Noggin polypeptide, such as NP005441.1 or the mature polypeptide contained therein) and SB431542 (collectively, “dual SMAD inhibitors”). Alternative factors (individually and/or in combination) could be used in the disclosed methods in place of either or both of the dual SMAD inhibitors, and/or be used in addition to one or both of these factors. Though these factors are sometimes referred to as “dual” SMAD inhibitors, more or fewer than two factors may be utilized within the scope of these methods. Other dual SMAD inhibitors are known such as, but not limited to, dorsomorphin.

Noggin is a secreted BMP inhibitor that reportedly binds BMP2, BMP4, and BMP7 with high affinity to block TGFβ family activity. SB431542 is a small molecule that reportedly inhibits TGFβ/Activin/Nodal by blocking phosphorylation of ACTRIB, TGFβR1, and ACTRIC receptors. SB431542 is thought to destabilize the Activin- and Nanog-mediated pluripotency network as well as suppress BMP induced trophoblast, mesoderm, and endodermal cell fates by blocking endogenous Activin and BMP signals. It is expected that agents having one or more of the aforementioned activities could replace or augment the functions of one or both of Noggin and SB431542, e.g., as they are used in the context of the disclosed methods. For example, it is envisioned that the protein Noggin and/or the small molecule SB4312542 could be replaced or augmented by one or more inhibitors that affect any or all of the following three target areas: 1) preventing the binding of the ligand to the receptor; 2) blocking activation of receptor (e.g., dorsomorphin), and 3) inhibition of SMAD intracellular proteins/transcription factors. Exemplary potentially suitable factors include the natural secreted BMP inhibitors Chordin (which blocks BMP4) and Follistatin (which blocks Activin), as well as analogs or mimetics thereof. Additional exemplary factors that may mimic the effect of Noggin include use of dominant negative receptors or blocking antibodies that would sequester BMP2, BMP4, and/or BMP7. Additionally, with respect to blocking receptor phosphorylation, dorsomorphin (or Compound C) has been reported to have similar effects on stem cells. Inhibition of SMAD proteins may also be affected using soluble inhibitors such as SIS3 (6,7-Dimethoxy-2-((2E)-3-(1-methyl-2-phenyl-1H-pyrrolo[2,3-b]pyridin-3-yl-prop-2-enoyl))-1,2,3,4-tetrahydroisoquinoline, Specific Inhibitor of Smad3, SIS3), overexpression of one or more of the inhibitor SMADs (e.g., SMAD6, SMAD7, SMAD 10) or RNAi for one of the receptor SMADs (SMAD1, SMAD2, SMAD3, SMAD5, SMAD8/9). Another combination of factors expected to be suitable for generating neural progenitors comprises a cocktail of Leukemia Inhibitory Factor (LIF), GSK3 inhibitor (CHIR 99021), Compound E (gamma secretase inhibitor XXI) and the TGFβ inhibitor SB431542 which has been previously shown to be efficacious for generating neural crest stem cells (Li et al., Proc Natl Acad Sci USA. 2011 May 17; 108(20):8299-304). Additional exemplary factors may include derivatives of SB431542, e.g., molecules that include one or more added or different substituents, analogous functional groups, etc. and that have a similar inhibitory effect on one or more SMAD proteins. Suitable factors or combinations of factors may be identified, for example, by contacting iPSCs with said factor(s) and monitoring for adoption of neural crest stem cell phenotypes or embryoid bodies, such as characteristic gene expression.

Following differentiation, the NPCs are isolated and collected for further processing. For example, the NPCs can be manually picked from lightly dissociated embryoid bodies (EBs) and placed in suspension (e.g., for about 5 days). The resulting NPCs are grown to confluence and then are incubated with dPBS for 2-10 minutes and then scraped and cultured to form neurospheres using neural growth factor (NGF). The neurospheres are then dissociated and replated and cultured (e.g., with constant shaking). Once the neurospheres are well formed, ROCK inhibitor or similar agent can be added, with the elimination of FGF from the media, and cultured for 1 to 3 days. The cells are then cultured without ROCK inhibitor in NG media without FGF for about 1 week. Astrocyte media is then added to the neurospheres for about 2 weeks and cultured while shaking at about 90 rpms. After about 2 weeks the spheres are plated and astrocytes project outside of the spheres to populate the plate surface.

It should also be noted that the iPSCs and NPC and astrocytes obtained herein can be cultured, expanded and used as cell banks. For example, once human induced pluripotent stem cells, NPCs or astrocytes have been established in culture, as described herein, they may be maintained or stored in cell “banks” comprising either continuous in vitro cultures of cells requiring regular transfer or cells which have been cryopreserved.

Cryopreservation of stem cells, or other cell of the disclosure, may be carried out according to known methods, such as those described in Doyle et al., (eds.), 1995, Cell & Tissue Culture: Laboratory Procedures, John Wiley & Sons, Chichester. For example, but not by way of limitation, cells may be suspended in a “freeze medium” such as, for example, culture medium further comprising 15-20% fetal bovine serum (FBS) and 10% dimethyl sulfoxide (DMSO), with or without 5-10% glycerol, at a density, for example, of about 4-10×10⁶ cells/ml. The cells are dispensed into glass or plastic vials which are then sealed and transferred to a freezing chamber of a programmable or passive freezer. The optimal rate of freezing may be determined empirically. For example, a freezing program that gives a change in temperature of −1° C./min through the heat of fusion may be used. Once vials containing the cells have reached −80° C., they are transferred to a liquid nitrogen storage area. Cryopreserved cells can be stored for a period of years, though they should be checked at least every 5 years for maintenance of viability.

The cryopreserved cells of the disclosure constitute a bank of cells, portions of which can be withdrawn by thawing and then used to produce a cell culture comprising stem cells, NPCs or astrocytes as needed. Thawing should generally be carried out rapidly, for example, by transferring a vial from liquid nitrogen to a 37° C. water bath. The thawed contents of the vial should be immediately transferred under sterile conditions to a culture vessel containing an appropriate medium. It is advisable that the cells in the culture medium be adjusted to an initial density of about 1-3×10⁵ cells/ml. Once in culture, the cells may be examined daily, for example, with an inverted microscope to detect cell proliferation, and subcultured as soon as they reach an appropriate density.

In another embodiment, cells of the disclosure (e.g., NPC, iPSCs etc.) can be obtained and banked. The banked cells can be autologous or allogeneic. The cells can be differentiated in vitro to glial restricted neural progenitor cells (GRNPCs), astrocytes or wherein glial restricted neural progenitor cells (GRNPCs) are delivered and differentiated in vivo to, for example, astrocytes.

Disclosed herein, in certain embodiments, is a method of treating a neurological condition associated with astrocyte or glial degeneration or mutations, comprising administering to the brain region of a subject in need thereof a composition comprising a glial restricted neural progenitor cells (GRNPCs).

In some embodiments, the non-neuronal cell is a glial restricted neural progenitor cells (GRNPCs), glial cell or an astrocyte.

The glial restricted neural progenitor cells (GRNPCs), glial cell or an astrocyte of the disclosure may be utilized in various therapeutic manners, for example, in a method for treating a neuronal cell disorder, disease or injury in a patient in need thereof, comprising administering to the patient a therapeutically effective amount of one or more compositions comprising isolated glial restricted neural progenitor cells (GRNPCs), glial cell or an astrocyte. For example, the disclosure contemplates transplanting GRNPCs made by a method of the disclosure into neural tissue of a subject having a neurodegenerative or neurological disorder that is associated with astrocyte dysfunction. Examples of neurodegenerative or neurological disorders that are associated with astrocyte dysfunction include, but are not limited to, Rett Syndrome, Alexander's disease, amyotrophic lateral sclerosis (ALS), epilepsy, Parkinson's disease, Alzheimer's disease, autism spectrum disorders and autism. In a particular embodiment, the neurodegenerative or neurological disorder that is associated with astrocyte dysfunction is Rett Syndrome.

In one embodiment of the disclosure, a subject is presented with GRNPC cells of the disclosure on an appropriate support material, such as an extracellular matrix or biocoated surface. The support material can be de novo or the support material utilized for culturing the GRNPC cells of the disclosure. For example, an exemplary of the disclosure comprises cultured GRNPCs on or in an extracellular and/or biocompatible matrix material. In an alternate embodiment, GRNPC of the disclosure are isolated from culture and presented to a subject in need thereof at a site where, for example, glial cells are needed. In one example, GRNPC of the disclosure lack mutations that cause a disease associated with the subject and are administered alone or in combination with other agents or treatment modalities. In more specific embodiments, the other agents are active agents. And in the most specific embodiments, the active agents include growth factors, cytokines, inhibitors, immunosuppressive agents, steroids, chemokines, antibodies, antibiotics, antifungals, antivirals, mitomycin C, or other cell types.

The disclosure provides a method of treating a subject having a neuronal disease associated with defective glial cell processing, the method comprising generating embryonic bodies (EBs) from ESCs or iPSCs by culturing the ESCs or iPSCs in a feeder-free maintenance medium and then in a SM1 neural induction medium; generating neural progenitor cells (NPCs) from EBs by plating the EBs onto laminin/polyornithine coated plates and culturing in SM1 neural induction media supplemented with bFGF; differentiating the NPCs to GRNPCs by culturing the EBs in microwell culture plates in SM1 neural induction media supplemented with bFGF to form cell aggerates; collecting and culturing the cell aggregates for at least 24 h in suspension and under agitation to form free-floating spheres; culturing the spheres in SM1 neural induction media supplemented with a ROCK inhibitor for at least 24 h, and then in SM1 neural induction media for at least 7 days; culturing the spheres in astrocyte growth media for at least 10 days to form astrospheres; and plating the astrospheres on laminin/polyornithine coated plates and culturing the plates for at least 5 days to form GRNPCs. In a further embodiment, the GRNPCs are banked in a tissue bank for later use. In still another or further embodiment, the GRNPCs are cultured and/or differentiated and screened with biological agents. In still another embodiment, the GRNPCs are transplanted into a patient or subject in need of GRNPCs, wherein the GRNPCs differentiate into normal glial cells that replace or supplant diseased or mutant glial cells. In another embodiment, the patient or subject has a disease or disorder associated with aberrant glial cells or glial cell activity.

The following examples are intended to illustrate but not limit the disclosure. While they are typical of those that might be used, other procedures known to those skilled in the art may alternatively be used.

### EXAMPLES

Serum-free GRNPCs production. Feeder-free control healthy hiPSCs were fed daily with mTeSR1. hiPSCs were passaged when they reached 80% confluence in colonies of 250-300 μm diameter. To generate embryonic bodies (EB), hiPSC cultures were dissociated using Accutase (Life Technologies) in PBS (1:1) for 10 minutes at 37° C. and centrifuged for 3 minutes at 150×g. The EBs were cultured in suspension in 6-well plates in mTeSR1 for 5 days; then switched to SM1 neural induction medium (DMEM/F12 supplemented with Neurocult SM1 Neuronal supplement, STEMCELL Technologies) with bFGF (20 ng/ml, Millipore Sigma) for 7 days. Thereafter, the EBs were plated onto laminin/polyornithine coated 6-well plates and cultured in SM1 media supplemented with bFGF. The plated spheres are then passaged 2-3 times with Accutase (Life Technologies) onto laminin/poly-ornithine coated 10 cm plates. At this stage, the NPCs (Neural Progenitor Cells) can be continuously passaged or frozen for future use. Once the NPCs reached 80-90% confluency they were dissociated using Accutase (Life Technologies) for 5 minutes at 37° C. and centrifuged for 5 minutes at 150×g. The cell pellet was resuspended in SM1 media supplemented with bFGF (20 ng/ml, Millipore Sigma). Approximately 3×10⁶ cells were transferred to one well of a 24-well Aggrewell 800 plate (STEMCELL Technologies) and centrifuged for 3 minutes at 100×g. After 24 hours, the cell aggregates were mechanically lifted via trituration and transferred to one well of a 6-well plate and kept in suspension under rotation (95 rpm) to form free-floating spheres. After 48 hours, the media was changed to SM1 media with 5 mM ROCK inhibitor (Y-27632; Calbiochem, Sigma-Aldrich, St. Louis, MO, USA). After another 48 hours, the media was changed to SM1 media for 10 days, replacing the SM1 media 2-3 times per week. Media was then changed to Astrocyte Growth Media (AGM, Astrocyte Growth Medium Bulletkit, Lonza Group, Basel, Switzerland) for 2 weeks, and then changed 2-3 times per week. Each well of spheres were then plated on laminin/polyornithine coated 10 cm plates and allowed to grow out onto the plate. After one week, the resulting GRNPCs were dissociated with Accutase for 5 minutes at 37° C. and used for transplantation.

Cortical Organoid co-culture. Feeder-free hiPSCs were fed daily with mTeSR1 (STEMCELL Technologies, Vancouver, Canada) for 7 days. Colonies were dissociated using Accutase (Life Technologies) in PBS (1:1) for 10 minutes at 37° C. and centrifuged for 3 minutes at 150×g. The cell pellet was resuspended in mTeSR1 supplemented with 10 mM SB431542 (SB; Stemgent, Cambridge, MA, USA) and 1 mM Dorsomorphin (Dorso; R&D Systems, Minneapolis, MN, USA). Approximately 3×10⁶ cells were transferred to one well of a 24-well Aggrewell 800 plate (STEMCELL Technologies) and centrifuged for 3 minutes at 100×g. After 24 hours, the cell aggregates were mechanically lifted via trituration and transferred to one well of a 6-well plate and kept in suspension under rotation (95 rpm) in the presence of 5 mM ROCK inhibitor (Y-27632; Calbiochem, Sigma-Aldrich, St. Louis, MO, USA) for 24 hours to form free-floating spheres. After 3 days, mTeSR1 was substituted by Medial [Neurobasal (Life Technologies) supplemented with GlutaMAX, 1% Gem21 NeuroPlex (Gemini Bio-Products), 1% N2 NeuroPlex (Gemini Bio-Products), 1% NEAA (Life Technologies), 1% PS (Life Technologies), 10 mM SB and 1 mM Dorso] for 7 days. Then, the cells were maintained in Media2 [Neurobasal with GlutaMAX, 1% Gem21 NeuroPlex, 1% NEAA and 1% PS] supplemented with 20 ng/ML FGF2 (Life Technologies) for 7 days, followed by 7 additional days in Media2 supplemented with 20 ng/ml of FGF2 and 20 ng/mL EGF (PeproTech, Rocky Hill, NJ, USA). Next, cells were transferred to Media3 [Media2 supplemented with 10 ng/ml of BDNF, 10 ng/mL of GDNF, 10 ng/ml of NT-3 (all from PeproTech), 200 mM L-ascorbic acid and 1 mM dibutyryl-CAMP (Sigma-Aldrich) to promote maturation, gliogenesis and activity]. After 7 days, cortical organoids were maintained in Media2 for as long as needed, with media changes every 3-4 days. After 14 days, individual cortical organoids are transferred to single wells of a 96-well round bottom plate (Corning Inc., Corning, New York, USA) in Media2. 20,000 GRNPCs in single cell suspension (as described in GRNPCs production) are added to each well and the plate is kept under rotation for four days, changing media once after two days. After four days, cortical organoids are transferred back to a 6-well plate for up to two months, changing media every 3-4 days.

Brain organoid readouts. Immunofluorescence and imaging analyses: brain cortical organoids were fixed with 4% paraformaldehyde for 48 hours at 4° C. Next, samples were permeabilized in 1×PBS (Corning) containing 0.1% (v/v) Triton X-100 for 10 minutes. Fixed cultures and sliced organoids were incubated with blocking solution for 1 hour [3% Bovine Serum Albumin (BSA); (Gemini) in 1×PBS]. Primary antibodies were diluted with blocking solution and incubated with sections overnight at 4° C. Sections were then washed twice with 1×PBS and incubated with the secondary antibody for 30 minutes at ambient temperature. Secondary antibodies (all conjugated to Alexa Fluor 488, 555 and 647) were purchased from Life Technologies and used at a 1:1000 dilution. After the 30 minutes incubation, samples were washed twice (1×PBS), incubated for 5 minutes with fluorescent nuclear DAPI stain (VWR; 1:10000), and mounted with Slow fade gold antifade reagent (Life Technologies). Images were blindly collected using an Axio Observer Z1 Microscope with ApoTome (Zeiss) and analyzed with ImageJ software.

Neuronal Soma size: neuronal soma size was quantified by tracing neurons in Neurolucida followed by unbiased counting using the Imaris software) as described in Marchetto et al. (Cell 143:527-539 (2010)) and Griesi-Oliveira et al. (Cell stem cell 25:558-569 e557 (2019)).

Synaptogenesis: the number of synapses was quantified by immunostaining post and pre-synaptic markers (Synapsin1, Psd-95, Homer1 and VGlut1), as described in Marchetto et al. and Griesi-Oliveira et al. The puncta quantification is performed using the Imaris software.

Network analyses: after plating brain organoids on MEAs, neural connectivity as quantified after evoked focal electrical stimulation and burst synchronization, spike frequency and pharmacology. The total number of bursts and its synchronization patterns data were extracted by the Mobius software followed by Neuroexplorer to create raster plots, as described in Vessoni et al. (Hum Mol Genet 25:1271-1280 (2016)) and Nageshappa et al. (Mol Psychiatry 21:178-188 (2016)).

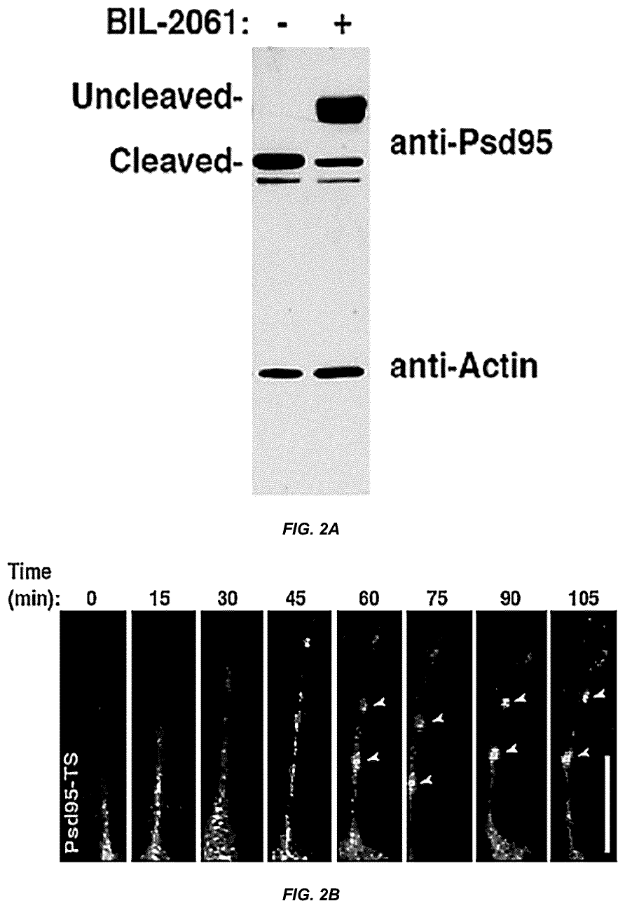

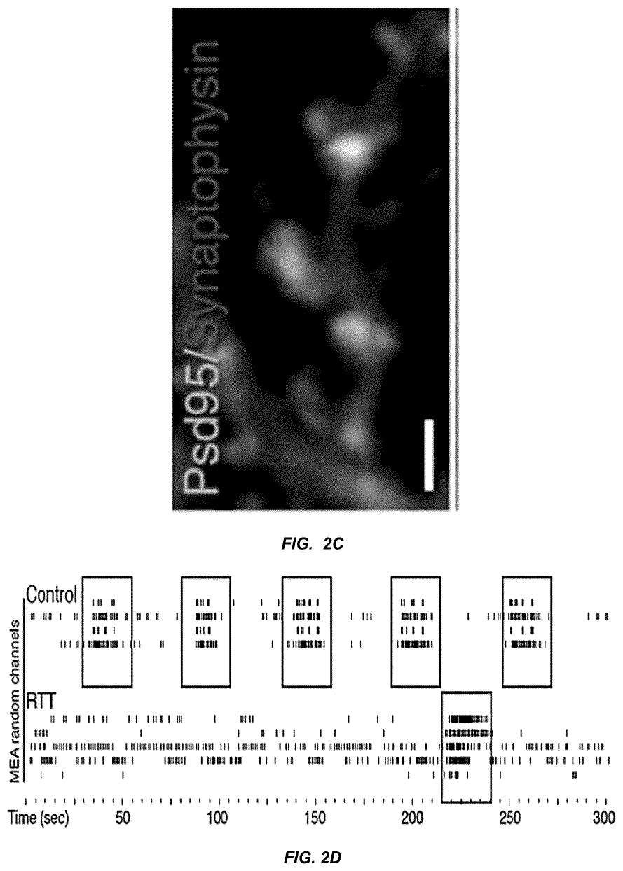

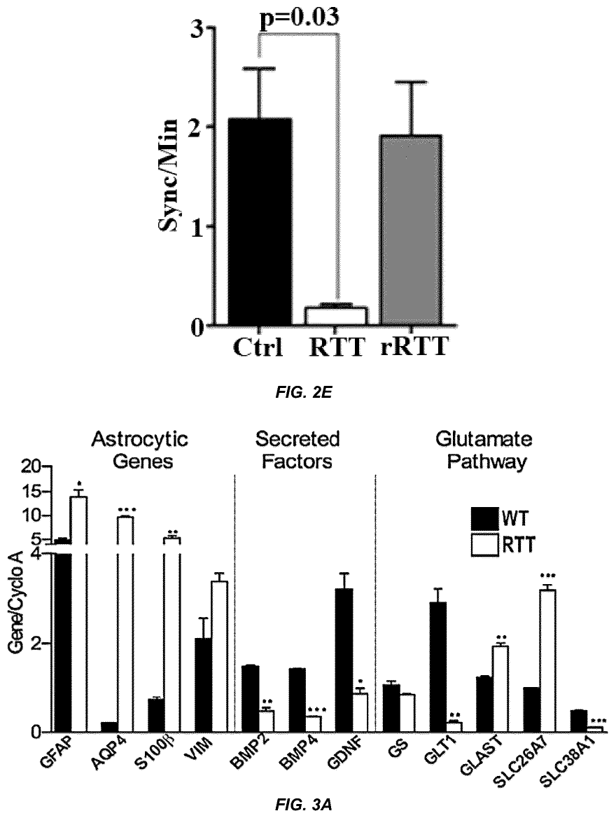

Preliminary Data. RTT-derived neurons have several phenotypes, including morphometric alterations (i.e., reduced branching and smaller soma size) and functional deficits (such as, lower number of excitatory synapses, abnormal calcium transients and decrease postsynaptic current frequency). These phenotypes have also been validated in iPSCs lines. The synaptogenesis was measured using a Psd95-GFP TIMESTAMP (TS) vector. This approach allows for a dynamic interpretation of synapse formation and not just a single time point analysis, discriminating between synapse maturation and developmental problems. The technology uses a covalent labeling of newly synthesized Psd95 with GFP reporter based on drug-dependent preservation of epitope tags. In this case, the protease inhibitor BIL-2061 was used to activate the process in culture, allowing the visualization of synapses in real time (see FIG. 2A-C). The deficiency in connectivity was further corroborated measuring neuronal network synchronization using multi-electrodes arrays (MEA). It was found that RTT-derived neuronal cultures lack burst synchronized episodes when compared to control (WT) cultures (see FIG. 2D-E). This neuronal network phenotype can be genetically rescued by correcting the respective MECP2 mutation in RTT neurons (see FIG. 2E).

Phenotypes for RTT astrocytes. Astrocytes were

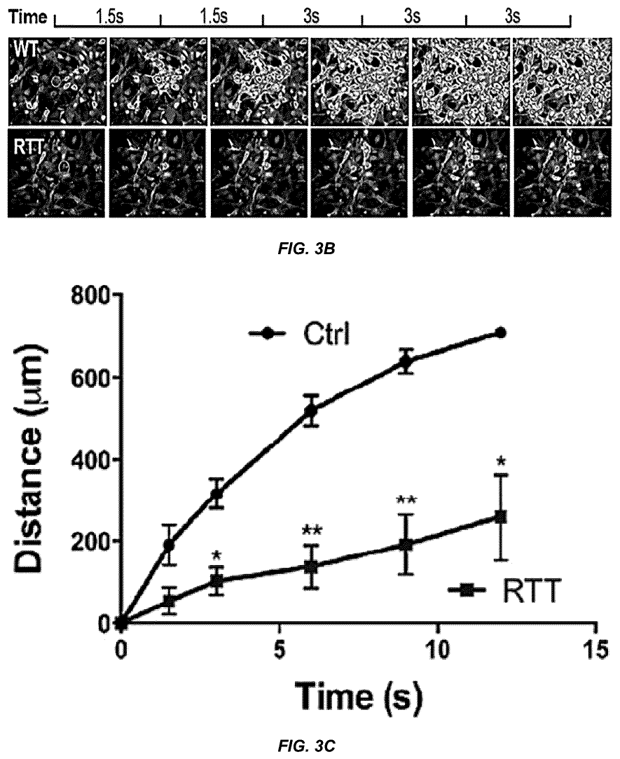

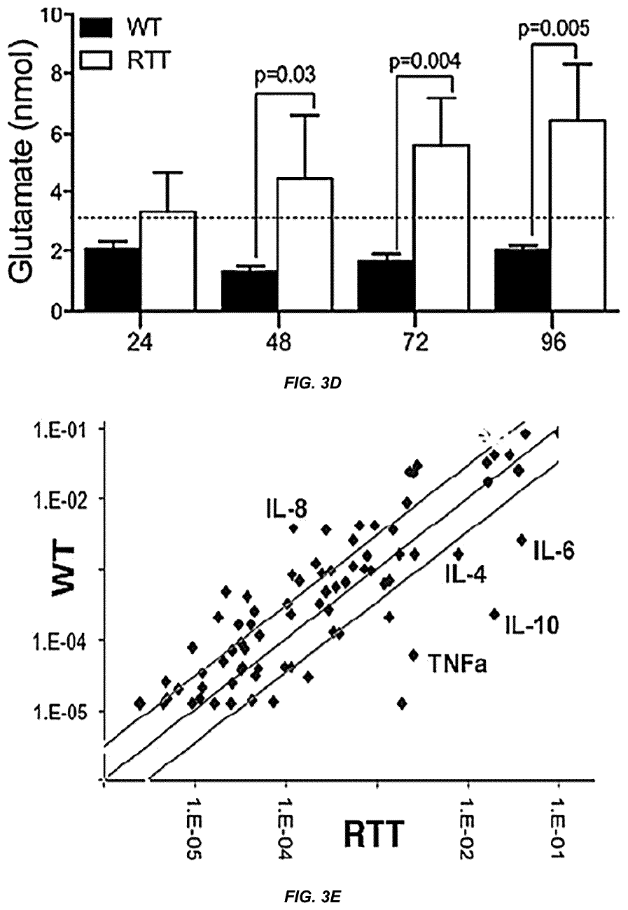

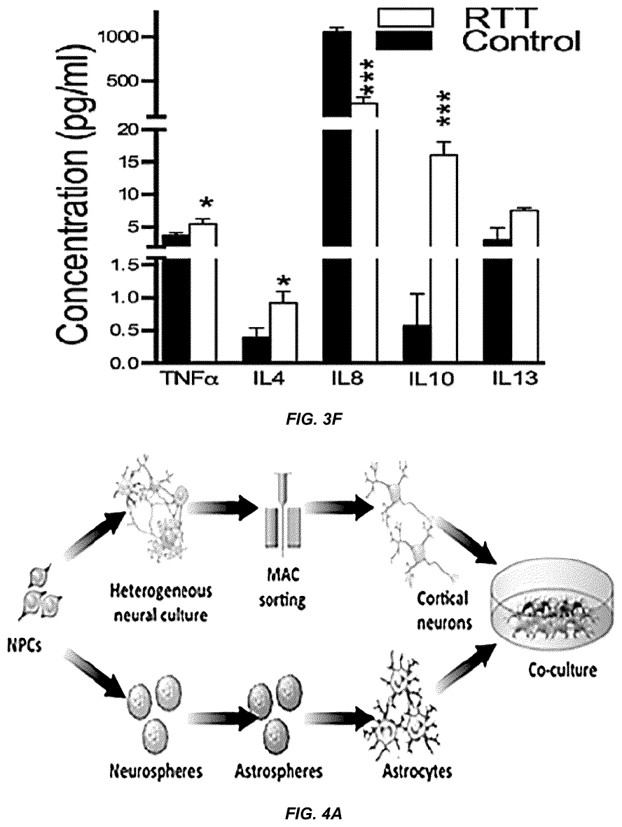

differentiated from both RTT and WT iPSCs and a phenotype in the gene expression was further investigated. Several genes were misregulated in RTT versus WT. The misregulated genes were validated independently, with a focus on genes known to be important for astrocyte function, such as GDNF (Glial Derived Neurotropic Factor) and aquaporin 4 (AQP4). The RTT-astrocytes also exhibited a lower expression of certain known secreted factors, such as Bone morphogenetic protein 2 and 4 (BMP2 and BMP4) and GDNF. The regulation of genes in the glutamate pathway was also affected in RTT cells (see FIG. 3A). In evaluating the function of the RTT astrocytes, it was observed that RTT astrocytes failed to propagate calcium waves after a mechanical stimulation of a single cell (see FIG. 3B-C). The ability of RTT astrocytes to clear glutamate from the cultures was also measured (see FIG. 3D). The data indicates that RTT astrocyte cultures accumulate glutamate in the media over time, indicating impairment in glutamate uptake/clearance mechanism. The cytokine secretion levels in RTT astrocytes were also evaluated. From an initial unbiased RNAseq gene expression analysis, cytokines were found to be the main metabolic pathway misregulated in RTT astrocytes. Whether RTT astrocytes would present an unbalanced cytokine signature compared to healthy astrocytes in the absence of any stimuli was further investigated. It was found that RTT astrocytes had high levels of basic, pro-inflammatory, intrinsic (no external stimuli were used) expression in comparison to controls. It was also found that several cytokines were upregulated in RTT astrocytes, including IL-6, IL-4, IL-10 and TNFα (see FIG. 3E-F).

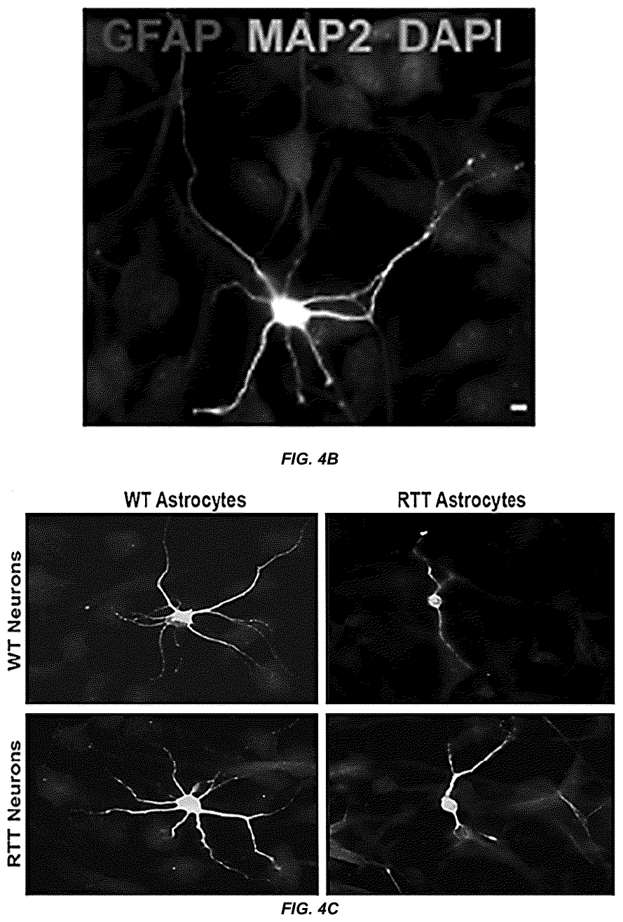

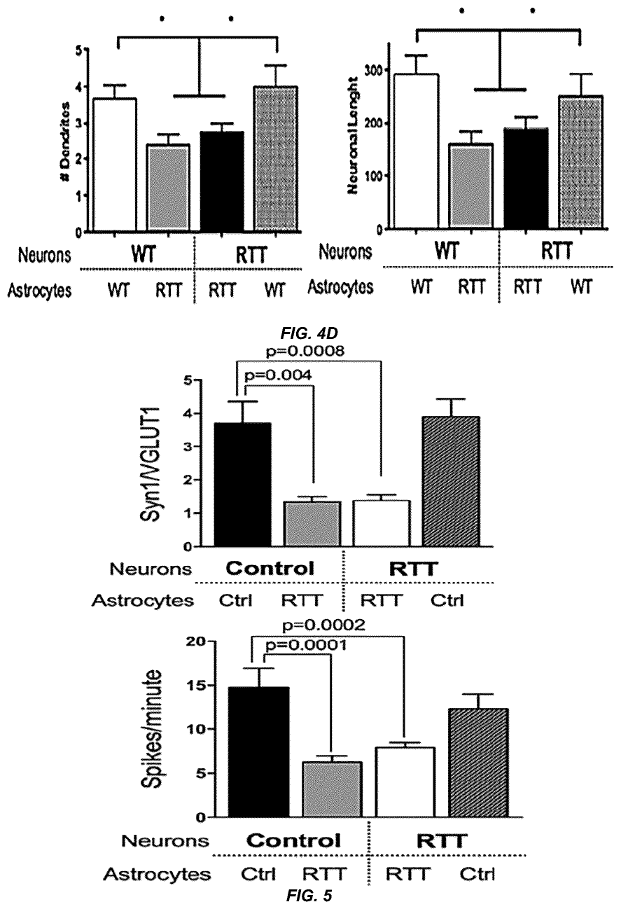

Astrocytes from non-affected individuals can rescue RTT neurons. Based on previous data, the hypothesis that RTT astrocytes would negatively impact co-cultured human neurons, while non-mutant, healthy, control astrocytes may help RTT neuronal functions. Plating iPSC-derived neurons on top of a well-characterized monolayer of astrocytes (positive labeling for GFAP, S100β and AQP4) (see FIG. 4A-B). The neuronal characterization was based on dendritic length, dendritic spine number, spine density, soma area, tree number and segments, obtained by immunofluorescence of fixed cells infected with syn1::EGFP and analyzed using the Neurolucida software. RTT astrocytes dramatically affected the morphology of the control neurons. In direct contrast, when RTT-derived neurons were plated with control astrocytes, most of these neuronal morphological phenotypes were rescued (see FIG. 4C-D). Moreover, the co-culture model allowed for the investigation of the co-localization of pre- and post-synaptic markers in neurons that are in contact with astrocytes. The preliminary results showed a significant decrease in the presence of synapsin 1 (syn1) protein levels in RTT neurons when they matured in the presence of RTT astrocytes. Co-localization of syn1 with excitatory vesicles of VGLUT1 protein was also decreased in RTT neurons compared to WT. This result reflects the misbalanced defects in synaptic vesicle proteins, especially in excitatory synapses in RTT neurons in the presence of RTT astrocytes (see FIG. 5). Furthermore, patch-clamping analysis from control neurons plated on top of control astrocytes showed characteristics of sodium and potassium currents in generating action potentials. In contrast, RTT astrocytes failed to induce neuronal maturation of sodium channels. Sodium currents were not detected and no action potential was obtained from 15 neurons that were tested using this model. RTT-derived neurons when co-cultured with healthy control astrocytes, were found to be functional. Network analyses using multi-electrode arrays (MEA) was also performed. Spikes were first measured when astrocyte-neuronal co-cultures set up on MEA plates were allowed to mature for an additional two weeks (see FIG. 5).

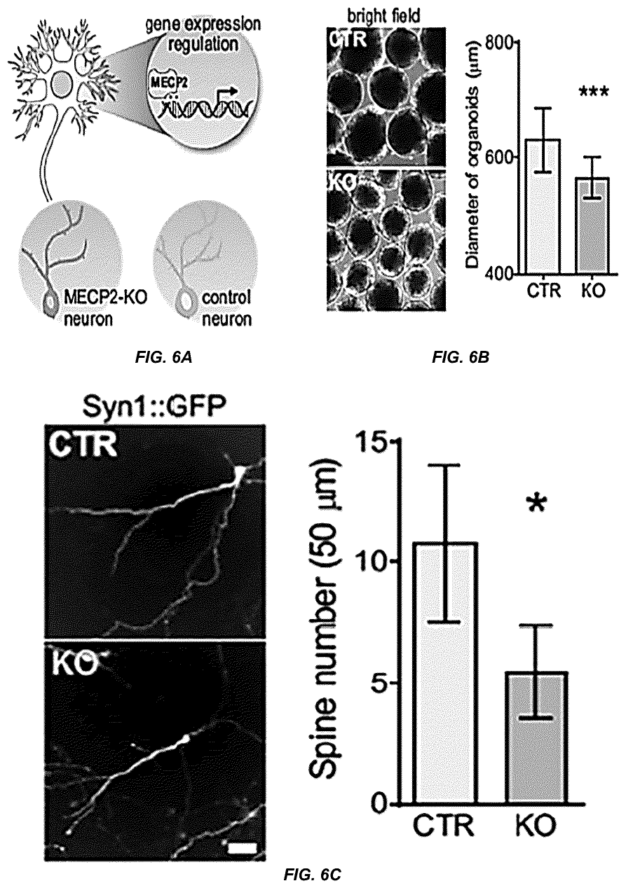

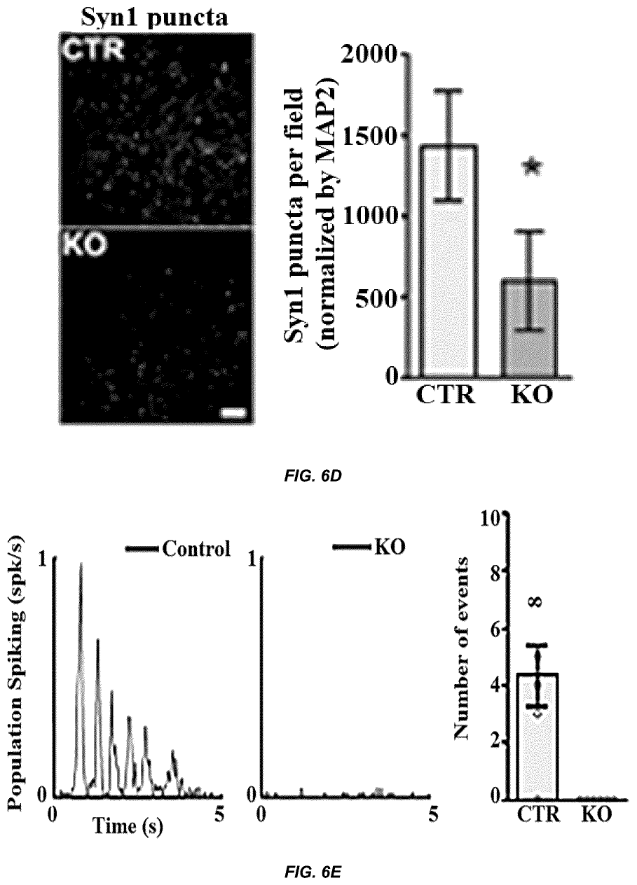

Brain organoids lacking MeCP2. A three-dimensional human neurodevelopmental model that closely recapitulates aspects of human fetal neurodevelopment was generated by using brain organoids that were derived from control iPSC lines where the MECP2 gene was disrupted by using CRISPR/Cas9. The morphological alterations in neuronal cells (soma size, dendritic arborization, etc.) were confirmed, and defects in neural networks were also observed (see FIG. 6).

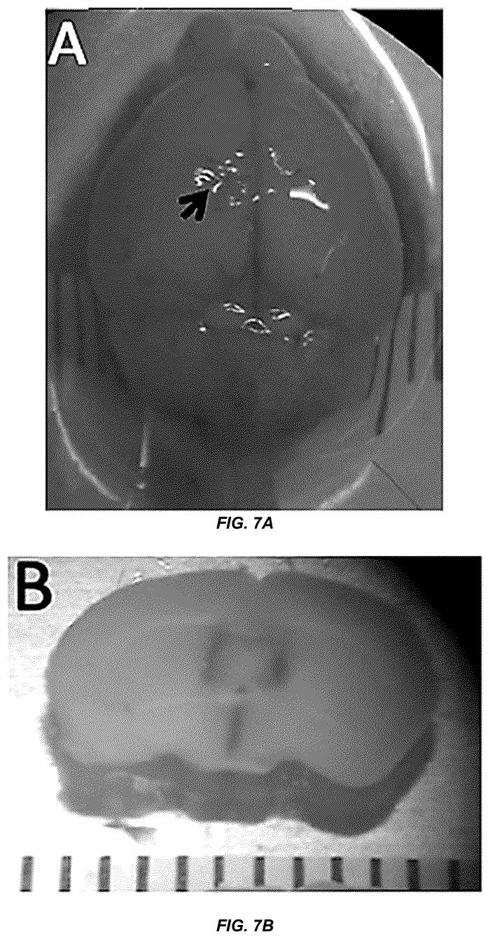

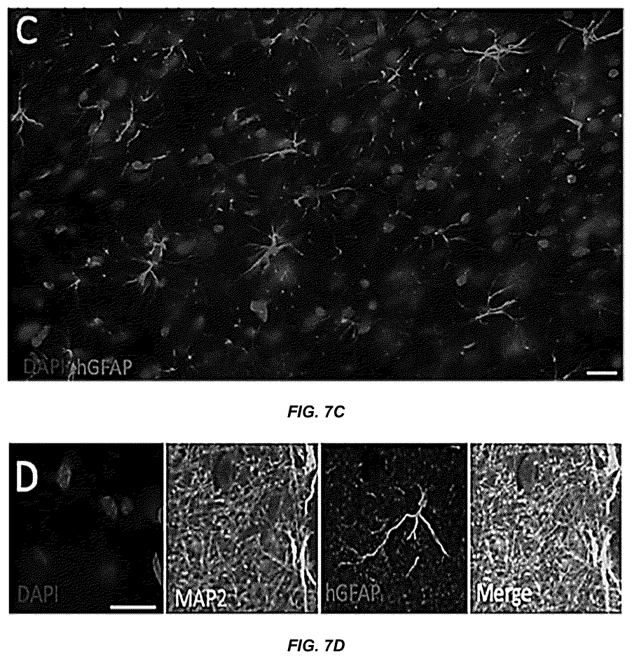

GRNPCs transplantation in mouse brains. As a proof-of-principle that the progenitor cells can integrate, migrate, differentiate and survive in the mouse brain for long periods, 80,000 GRNPCs were transplanted via bilateral injection into the corpus callosum of mouse brains as described in Wang et al. (Cell Mol Neurobiol 32:373-380 (2012)), Benraiss et al. (Nat Commun 7:11758 (2016)), and Windrem et al. (J Neurosci 34:16153-16161 (2014)). At 3 months of age, transplanted mice were anesthetized with isoflurane, then perfused with cold PBS. Brains were extracted and fixed for 24 hours in cold 4% paraformaldehyde. The initial experiments with wild-type mice indicated that transplanted cells could successfully differentiated into astrocytes and were found everywhere in the mouse brain after 3 months. Similarly to what previously described, human astrocytes were observed to outcompete the mouse astrocytes in vivo over time, taking over almost the entire mouse brain. A human-specific GFAP antibody was used to visualize human cells in the mouse brain (see FIG. 7). There was no detection of any other cell fate (NPCs, neurons, microglia or oligodendrocytes) from the transplantation experiments after a month.

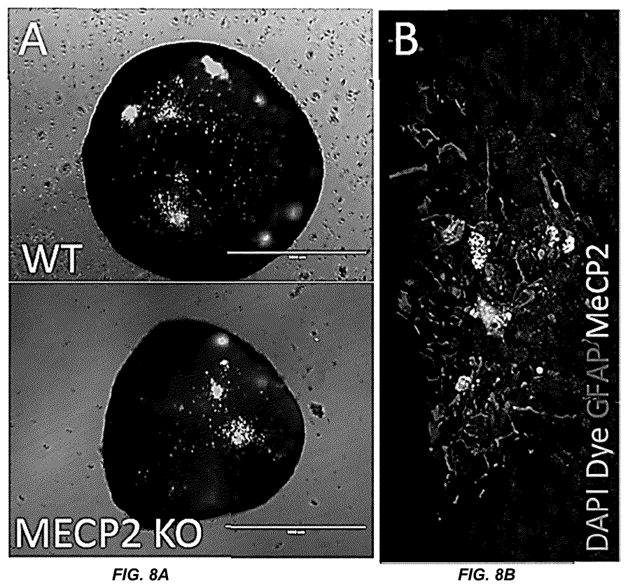

GRNPCs transplantation in brain organoids. 20,000 dyed GRNPCs in suspension were added to single unaffected (WT) and MECP2 KO brain organoids at 1 month of age in order to demonstrate that GRNPCs can also integrate, proliferate, and differentiate in a human brain development milieu. At this stage, brain organoids are composed mainly of NPCs and neurons. Thus, all astrocytes result from the transplantations. 30 days after transplantation, the brain organoids were fixed in cold 4% paraformaldehyde for 48 hours and sectioned for immunohistochemical analysis. The experiments demonstrated successful engraftment and survival of GRNPCs. The transplanted cells also integrated and differentiated into mature astrocytes (see FIG. 8A-B).

It will be understood that various modifications may be made without departing from the spirit and scope of this disclosure. Accordingly, other embodiments are within the scope of the following claims.

1. A method to make glial restricted progenitor cells (GRNPCs) comprising:
generating embryonic bodies (EBs) from ESCs or iPSCs by culturing the ESCs or iPSCs in a feeder-free maintenance medium and then in a SM1 neural induction medium;
generating neural progenitor cells (NPCs) from EBs by plating the EBs onto laminin/polyornithine coated plates and culturing in SM1 neural induction media supplemented with bFGF;
differentiating the NPCs to GRNPCs by:
culturing the EBs in microwell culture plates in SM1 neural induction media supplemented with bFGF to form cell aggerates;
collecting and culturing the cell aggregates for at least 24 h in suspension and under agitation to form free-floating spheres;
culturing the spheres in SM1 neural induction media supplemented with a ROCK inhibitor for at least 24 h, and then in SM1 neural induction media for at least 7 days;
culturing the spheres in astrocyte growth media for at least 10 days to form astrospheres;
plating the astrospheres on laminin/ polyornithine coated plates and culturing the plates for at least 5 days to form GRNPCs.

2. The method of claim 1, wherein the ESCs or iPSCs are human ESCs or human iPSCs.

3. The method of claim 2, wherein the human ESCs or human iPSCs are from a subject who does not have a neurological or neurodegenerative disorder.

4. The method of claim 3, wherein the neurological or neurodegenerative disorder is Rett Syndrome.

5. The method of claim 1, wherein the feeder-free maintenance medium comprises DMEM/F12, FGF, TGFβ1, pipecolic acid, GABA, LiCl, lipid concentrate, L-glutamine-BME, MEM NEAA, and NaHCO₃.

6. The method of claim 1, wherein the SM1 neural induction media comprises DMEM/F12, insulin, transferring, progesterone, putrescine, selenium, vitamins, fatty acids, glutathione, pyruvate, catalase and superoxide dismutase.

7. The method of claim 1, wherein the ROCK inhibitor is selected from Y-30141, Y-33075, Y-39983, TCS-7001, verosudil, thiazovivin, RKI-1447, LX-7101, H-1152, GSK-576371, AT-13148, BA-210, and Y-27632.

8. The method of claim 7, wherein the ROCK inhibitor is Y-27632.

9. The method of claim 1, wherein the Astrocyte Growth Media comprises basal media supplemented with growth factors, cytokines, and supplements for Astrocyte growth.

10. A pure or substantially pure population of GRNPC obtained by the method of claim 1.

11. A composition comprising a pharmaceutically acceptable carrier and the substantially pure population of claim 10.

12. A method of treating a subject having a neurodegenerative or neurological disorder comprising:
transplanting the substantially pure population of claim 10 into neural tissue of a subject having a neurodegenerative or neurological disorder that is associated with astrocyte dysfunction.

13. The method of claim 12, wherein the neurodegenerative or neurological disorder that is associated with astrocyte dysfunction is selected from the group consisting of Rett Syndrome, Alexander's disease, amyotrophic lateral sclerosis (ALS), epilepsy, Parkinson's disease, Alzheimer's disease, and autism.

14. The method of claim 13, wherein the neurodegenerative or neurological disorder that is associated with astrocyte dysfunction is Rett Syndrome.

15. A method for treating a subject with a disease associated with malfunctioning astrocytes or glial cells, wherein the method comprises:
transplanting the cell population of claim 10 to an affected neuronal location of a subject, wherein the transplanted cells rescue or restore glial cell and/or neuronal function,
thereby treating the subject with the disease associated with malfunctioning glial cells.

16. The method of claim 15, wherein the subject is a mammal.

17. The method of claim 16, wherein the mammal is human, rat, dog, cat, pig, horse, rabbit, cow, monkey or mouse.
